(19) United States
(12) Patent Application Publication
(10) Pub. No.: US 2014/0261463 A1
(43) Pub. Date: Sep. 18, 2014

(54) Endotrachael Tube Holding Device with Bite Block

(71) Applicant: Hollister Incorporated, Libertyville, IL (US)

(72) Inventors: Peter L. Visconti, Gurnee, IL (US); Brian T. Leadingham, Pleasant Prairie, WI (US); Patrick C. Tetzlaff, Racine, WI (US); Paola M. Wisner, Des Planes, IL (US); Richard J. Hantke, Chicago, IL (US); Meagan R. Shanahan Pheil, Chicago, IL (US); Davis A. Knauz, Riverwoods, IL (US); Cheryl D. Berger, Round Lake, IL (US); Geroge J. Cisko, Spring Grove, IL (US); Thai H. Dang, Gurnee, IL (US); Thomas H. Gilman, Spring Grove, IL (US); Noah K. Meade, Grayslake, IL (US); Tze Wan Pansy Chung, Fox River Grove, IL (US); Christina Augustyn, Chicago, IL (US); Daniel A. March, Lake Villa, IL (US); David McDonough, Pleasant Prarie, WI (US); James Brandon Barker, Antioch, IL (US)

(73) Assignee: Hollister Incorporated, Libertyville, IL (US)

(21) Appl. No.: 14/213,695

(22) Filed: Mar. 14, 2014

Related U.S. Application Data
(63) Continuation-in-part of application No. 13/840,375, filed on Mar. 15, 2013.

(51) Int. Cl. A61M 16/04

(52) U.S. Cl. CPC A61M 16/0493
USPC 128/861

(57) ABSTRACT

A bite block for an endotracheal tube has a tubular wall with a pair of opposed ends, a central opening along a length of the bite block between the pair of opposed ends, an interior wall surface, and an accessory line channel positioned between the interior wall surface and an endotracheal tube extending through the central opening. The accessory line channel is formed by a wall segment that is recessed into the interior wall surface, the wall segment being thinner than a thickness of the tubular wall adjacent the wall segment. The bite block can be part of or attachable to an endotracheal tube holding device attachable to a patient.

### RELATED APPLICATION DATA

This patent is a continuation-in-part of and claims priority benefit of co-pending U.S. patent application Ser. No. 13/840,375 filed Mar. 15, 2013 and entitled “Endotracheal Tube Holding Device with Bite Block.” This prior filed application is hereby incorporated by reference herein in its entirety.

### BACKGROUND

1. Field of the Disclosure

The present invention is generally directed to medical tube devices, and more particularly to a device for holding an endotracheal tube on a patient and having a bite block integrated with the device.

2. Description of Related Art

Endotracheal (ET) tubes are commonly inserted through the mouth and into the trachea of patients under critical care. The ET tube is used to maintain an open airway for the patient to breathe and to allow mechanical assistance of breathing. ET tubes are often placed prior to surgery or are used on trauma or critically ill patients that may require intubation for extended periods of time. Many instances in which a patient is intubated require that the tube remain in place for approximately 48 to 72 hours and, in some circumstances, the period of use may be extended for 7 to 14 days or more.

There are many known methods and devices for securing an ET tube on a patient. One such device is manufactured and sold by Hollister, Inc., the assignee of the present patent under the name of Anchor Fast. The Anchor Fast product has a track that is secured to a patient via a head strap. The track is connected to cheek plates with cheek pads that are skin friendly and that have adhesive patches to help retain the track in the proper position on the patient's face during use. A tube holder is side-to-side adjustable on the track and is used to secure an ET tube to the device.

An earlier version of the Anchor Fast ET tube holder is disclosed in U.S. Pat. No. 5,490,504 to Hollister, Inc. The '504 patent discloses a device that has the track and tube holder, including side-to-side adjustability of the tube holder and ET tube that is secured thereto. However, the '504 patent does not disclose inclusion of the cheek pads and cheek plates.

One problem with these and other known ET tube holding devices is that the patient may sometimes bite down hard or clamp onto the ET tube with their teeth or gums (denture wearers). This can restrict or close off the airway within the tube. Some manufacturers have developed stand-alone bite blocks that can be attached to the ET tube and positioned between the patient's teeth during use. These types of bite block products must be separately manufactured, distributed, stored, and installed. These types of bite blocks are positioned around the ET tube and are intended to protect the ET tube and inhibit the patient from crushing of the ET tube. Others have attempted to develop air assist breathing devices and even ET tube holders that incorporate a bite block function into the product. These known products are generally large, cumbersome, and complicated devices that can be difficult to manipulate and install. Medical personnel have not heretofore taken to these types of integrated products.

Another problem with these types of ET tube holding devices is that the tube often is accompanied by one or more accessory lines that also pass into the patient. For example, many ET tubes have an inflation cuff on the end of the tube that is inserted into the patient's throat. The cuff is inflated after insertion of the tube to help retain the ET tube in the patient's trachea. Typically, a pilot line is coupled to the cuff and extends along the outside of the ET tube. The pilot line routing must be taken into account so that the line is not crushed or pinched off by any part of the holding device or the ET tube. This is particularly true where a bite block function is provided. Also, a subglottic suction line is sometimes used to suction subglottic secretions in the patient's airway. The subglottic suction line typically runs adjacent the ET tube when inserted. The line must also not be crushed or pinched off.

### SUMMARY

In one example according to the teachings of the present invention, a device for holding an endotracheal tube to a patient has a track configured to fit adjacent a lip on a patient's face. The track has a face contacting side, an exposed side opposite the face contacting side, and a pair of opposite ends. A tube holder is coupled to and slidable along the track between the opposite ends. A positioning mechanism is releasably lockable to allow selective lateral repositioning of the tube holder and an endotracheal tube held thereby along the track and to retain the tube holder at a selected position along the track. A bite block is carried by the tube holder and is slidable therewith along the track. The bite block has a tubular wall, a central opening along a length of the bite block, an interior wall surface, and a pair of opposite open ends. The bite block is positioned spaced vertically from the track. One of the opposite ends if the bite block is a working end portion that extends in the direction of and further beyond the face contacting side of the track. An accessory line channel is positioned between the interior wall surface and an endotracheal tube that extends through the central opening.

In one example, the bite block can have an insertion slot through the tubular wall and along the entire length of the bite block. The tubular wall can be sufficiently flexible to allow an endotracheal tube to be inserted laterally into the central opening through the insertion slot.

In one example, an insertion slot can be defined between spaced apart free edges of the tubular wall that confront one another along the length of the bite block.

In one example, an insertion slot can be defined between spaced apart free edges of the tubular wall along a length of the bite block. Each of the free edges can have a flange extending inward into the central opening of the bite block. Each flange can be configured to close against and engage the other flange if a sufficient clamping force is exerted on an outer surface of the bite block.

In one example, the bite block can have two of the accessory line channels, one each formed along the interior wall surface adjacent each of two flanges along free edges of an insertion slot and within the central opening of the bite block.

In one example, an insertion slot can be defined between spaced apart free edges of the tubular wall along the length of the bite block. At least one of the free edges can have a flange extending inward into the central opening of the bite block whereby the accessory line channel is formed along the interior wall surface adjacent the flange.

In one example, the bite block can have a living hinge or relief in the tubular wall generally opposite an insertion slot of the bite block and extending a length of the bite block.

In one example, the bite block can have a blind slot formed in the tubular wall of the bite block and extending only part way along the length of the tubular wall and positioned generally opposite an insertion slot in the tubular wall.

In one example, a blind slot can be formed in either the working end or the other opposite end, or both, of the tubular wall of the bite block.

In one example, the accessory line channel can be formed between two spaced apart ribs extending the length of the bite block on the interior wall surface.

In one example, a wall segment of the tubular wall can be between two spaced apart ribs on the interior wall surface of the tubular wall and the wall segment can be thinner than a thickness of at least a substantial portion of the remaining parts of the tubular wall.

In one example, the accessory line channel can be formed between two spaced apart ribs extending the length of the bite block on the interior wall surface. A wall segment can be between the two spaced apart ribs and can be thinner than a thickness of at least a substantial portion of the remaining parts of the tubular wall. The two spaced apart ribs and the wall segment can be on the bottom side of the tubular wall and the wall segment can be is recessed into the interior wall surface and can be thinner than a thickness of the tubular wall adjacent the wall segment.

In one example, the accessory line channel can be formed by a wall segment that is recessed into the interior wall surface. The wall segment can be thinner than a thickness of the tubular wall adjacent the wall segment.

In one example, the accessory line channel can be formed by a wall segment that is recessed into the interior wall surface. The wall segment can be thinner than a thickness of the tubular wall adjacent the wall segment and can be a V-shaped groove extending along and recessed into the inner surface of the wall.

In one example, the device can include a cheek plate connected to each of the opposite ends of the track, a cheek pad coupled to each of the cheek plates, an adhesive layer on a face contacting side of each pad, and an adjustable head strap coupled to the device for securing the device to a patient's head and retaining the track, cheek plates, and cheek pads on the patient's face.

In one example, the bite block can be integrally molded as a part of the tube holder or another part of the endotracheal tube holding device.

In one example, the bite block can be selectively attachable to and can be optionally detachable from the tube holder or another part of the endotracheal tube holding device.

In one example according to the teachings of the present invention, a bite block for an endotracheal tube can have a tubular wall with a pair of opposed ends, a central opening along a length of the bite block between the pair of opposed ends, an interior wall surface, and an accessory line channel positioned between the interior wall surface and an endotracheal tube extending through the central opening. The accessory line channel is formed by a wall segment that is recessed into the interior wall surface. The wall segment can be thinner than a thickness of the tubular wall adjacent the wall segment.

### BRIEF DESCRIPTION OF THE DRAWINGS

Objects, features, and advantages of the present invention will become apparent upon reading the following description in conjunction with the drawing figures, in which:

FIG. 1 shows a front and side perspective view of one example of a device for holding or securing an endotracheal tube, the device constructed in accordance with the teachings of the present invention and including a tube holder centered on the device.

FIG. 2 shows a perspective cross-section taken along line 2-2 of the device shown in FIG. 1.

FIG. 3 shows an alternate top, front, and side perspective view of the device shown in FIG. 1 and with the tube holder moved to a position offset from the centered position.

FIG. 4 shows a cross-section taken along line 4-4 of the device shown in FIG. 3.

FIG. 5 shows a rear and side perspective view of only the tube holder portion of the device shown in FIG. 1.

FIG. 6 shows a front view of the tube holder shown in FIG. 5

FIG. 7 shows a rear view of the tube holder shown in FIG. 5.

FIG. 8 shows a right side view of the tube holder shown in FIG. 5.

FIG. 9 shows a top view of the tube holder shown in FIG. 5.

FIG. 10 shows a left side view of the tube holder of FIG. 5.

FIG. 11 shows a bottom view of the tube holder of FIG. 5.

FIG. 12 shows a rear and right perspective view of one alternative example of a tube holder for the device shown in FIG. 1.

FIG. 13 shows a rear view of the tube holder of FIG. 12.

FIG. 14 shows a rear and right perspective view of another alternative example of a tube holder for the device shown in FIG. 1.

FIG. 15 shows a rear view of the tube holder of FIG. 14.

FIG. 16 shows a rear and left side perspective view of another alternative example of a tube holder for the device shown in FIG. 1.

FIG. 17 shows a rear view of the tube holder of FIG. 16.

FIG. 18 shows a rear and left side perspective view of another alternative example of a tube holder for the device shown in FIG. 1.

FIG. 19 shows a rear view of the tube holder of FIG. 18.

FIG. 20 shows a rear and left side perspective view of another alternative example of a tube holder for the device shown in FIG. 1.

FIG. 21 shows a rear view of the tube holder of FIG. 20.

FIG. 22 shows a rear and left side perspective view of another alternative example of a tube holder for the device shown in FIG. 1.

FIG. 23 shows a rear view of the tube holder of FIG. 22.

FIG. 24 shows a rear and left side perspective view of another alternative example of a tube holder for the device shown in FIG. 1.

FIG. 25 shows a rear view of the tube holder of FIG. 24.

FIG. 26 shows a rear and left side perspective view of another alternative example of a tube holder for the device shown in FIG. 1.

FIG. 27 shows a rear view of the tube holder of FIG. 24.

FIG. 28 shows a rear and left side perspective view of another alternative example of a tube holder for the device shown in FIG. 1.

FIG. 29 shows a rear view of the tube holder of FIG. 28.

FIG. 30 shows a front and left side perspective view of the tube holder of FIG. 28 in use on a device like that in FIG. 1.

FIG. 31 shows a rear and left side perspective view of another alternative example of a tube holder for the device shown in FIG. 1.

FIG. 32 shows a rear view of the tube holder of FIG. 31.

FIG. 33 shows a front and left side perspective view of the tube holder of FIG. 31 in use on a device like that in FIG. 1.

FIG. 34 shows a rear and left side perspective view of another alternative example of a tube holder for the device shown in FIG. 1.

FIG. 35 shows a rear view of the tube holder of FIG. 34.

FIG. 36 shows a front and left side perspective view of the tube holder of FIG. 34 in use on a device like that in FIG. 1.

FIG. 37 shows a rear and left side perspective view of another alternative example of a tube holder for a device like that shown in FIG. 1.

FIG. 38 shows a front and left side perspective view of another alternative example of a tube holder for a device like that in FIG. 1.

FIG. 39 shows a rear view of the bite block of the tube holder of FIG. 38.

FIG. 40 shows a side view of another alternative example of a tube holder and integrated bite block for a device like that in FIG. 1.

FIG. 41 shows a side view of another alternative example of a tube holder and integrated bite block for a device like that in FIG. 1.

FIG. 42 shows an end view of another alternative example of a bite block for a device like that in FIG. 1.

FIG. 43 shows an end view of another alternative example of a bite block for a device like that in FIG. 1.

FIG. 44 shows an end view of another alternative example of a bite block for a device like that in FIG. 1.

FIG. 45 shows an end view of another alternative example of a bite block for a device like that in FIG. 1.

FIG. 46 shows an end view of another alternative example of a bite block for a device like that in FIG. 1.

FIG. 47 shows a side view of another alternative example of a tube holder and integrated bite block for a device like that in FIG. 1.

FIG. 48 shows an end view of the tube holder and bite block shown in FIG. 47.

FIG. 49 shows a side view of another alternative example of a tube holder and a separately attachable bite block for a device like that in FIG. 1 and with the bite block detached.

FIG. 50 shows the tube holder and bite block of FIG. 49 but attached to one another.

FIG. 51 shows a side view of another alternative example of a tube holder and a separately attachable bite block for a device like that in FIG. 1 and with the bite block detached.

FIG. 52 shows an end view of the bite block of FIG. 51.

FIG. 53 shows the tube holder and bite block of FIG. 49 but attached to one another.

FIG. 54 shows a side view of another alternative example of a tube holder and an intermediate joint for a device like that in FIG. 1 and with the intermediate joint part detached and configured for attaching in a separate bite block to the tube holder.

FIG. 55 shows a perspective view of another example of a bite block and configured for attachment to the tube holder and intermediate joint of FIG. 54.

FIG. 56 shows a perspective view of the intermediate joint of FIG. 54.

FIG. 57 shows a perspective view of the bite block of FIG. 55 attached to the intermediate joint part of FIG. 56.

### DETAILED DESCRIPTION OF THE DISCLOSURE

The disclosed ET tube holding devices solve or improve upon one or more of the above-noted and/or other problems and disadvantages with prior know ET tube holding devices. In one example, the disclosed devices have a bite block integrated into a tube holder portion of the device. In one example, the disclosed devices have a tube holder that is side-to-side adjustable along with an ET tube secured by the device and a bite block integrated into the tube holder. In one example, the disclosed devices have a bite block that is integrally molded as a part of the tube holder. In one example, the disclosed devices have a bite block with one or more features to assist in inserting an ET tube, retaining the ET tube in position once inserted, and accommodating an accessory line that passes within the bite block but outside of the ET tube. These and other objects, features, and advantages of the present invention will become apparent to those having ordinary skill in the art upon reading this disclosure.

Turning now to the drawings, FIGS. 1-4 show one example of a device 30 for holding and securing a tube, such as an ET tube, on a patient. Many aspects of the device are disclosed in detail in the aforementioned U.S. Pat. No. 5,490,504. The entire content of the '504 patent is hereby incorporated by reference herein. As shown in FIG. 1, the device 30 is an ET tube attachment device for securing an ET tube to a patient requiring critical medical care. The disclosed device 30 generally has a track 32 that is configured to fit adjacent a lip on a patient's face. In this particular example, the track 32 is configured to rest above the patient's upper lip. However, the track 32 can instead be configured to rest below a patient's lower lip in another example. In each example, however, the track 32 is intended to extend laterally or lie horizontally across a portion of the user's face. The track 32 has a face contacting side 34 and an exposed side 36 opposite the face contacting side. The track 32 also has a pair of opposite ends 38.

The device 30 also generally has a tube holder 40 that is coupled to and slidable along the track 32 between the opposite ends 38 on the track. The device 30 also has a positioning mechanism 42 that is releasably lockable to allow selective lateral repositioning of the tube holder 40, as well as an endotracheal tube 44 that is held or secured thereby, along the track 32. The positioning mechanism 42 is also configured to retain the tube holder 40 at a selected position along the track 32. The device 30 also incorporates a bite block 46 that is carried by the tube holder 40 and slidable therewith along the track 32. Details of the bite block 46 are described in greater detail below. The bite block 46 in this example is positioned and spaced vertically below the track 32 so that the bite block extends between the teeth within a patient's mouth during use. If the track 32 were instead positioned below a patient's lower lip, the bite block 46 would be spaced vertically above the track.

The track 32 is preferably made of a tough, durable, semi-rigid but somewhat flexible plastic material, such as polyethylene, and is preformed or molded with a curved or arcuate shape to fit on a region adjacent to and along one lip of the patient. Although track 32 can be positioned along either of the patient's lips, it may be preferable to position the track along the patient's upper lip to avoid having movement of the patient's jaw affect positioning and performance of the device 30, and particularly the ET tube 44.

The device 30 in this example also has a cheek plate 50 connected to each of the opposite ends 38 of the track 32. A skin friendly cheek pad 52 is also coupled to each of the cheek plates 50 on the inside face of each plate. An adhesive layer (not show) can be provided on the face contacting side 54 of each of the cheek pads 52. The adhesive layer can also be skin friendly and can help adhere the cheek plates 50 and track 32 to the patient's face during use. The cheek plates 50 and pads 52 can be curved or contoured to closely follow the curved contour of a patient's face. Each cheek plate 50 in this example can have one or more strap loops 56 at or near their free ends. An adjustable head strap 58 can be coupled to the device 30 via the strap loops 56 for securing the device to a patient's head and retaining the track 32, cheek plates 50, and cheek pads 56 on the patient's face. A separate lip pad 58 can be provided on the face contacting side 34 of the track 32 as well. The lip pad can be adhered to the track and can also have a skin friendly adhesive on the rear exposed side to help retain the track in position against the patient's face during use.

As will be evident to those having ordinary skill in the art, the track, cheek plates, cheek pads, and head strap can vary in configuration and construction and yet fall within the scope of the invention and claims. The track and cheek plates can be molded as one integrated plastic structure, if desired. The head strap can be formed having any suitable adjustable fastening mechanism, such as a hook and loop structure on a fabric strap. The cheek plates can be formed having any number of configurations and constructions and can utilize a minimum amount of base material (i.e., plastic) and yet function as intended.

With reference to FIGS. 5-11, the tube holder 40 generally has a shuttle 60 that is slidably mounted on the track 32. The tube holder 42 also has an arm 62 that extends in a direction perpendicular to the track 32 and in a direction away from the exposed or outer surface 36 of the track. Securement means are provided on the arm 62 for securing an ET tube 44 thereto in a direction parallel to the arm. In one example, the securement means can employ a soft, flexible, elongate tube strap 64 of an elastomeric material. One end of the tube strap 64 has an enlarged retaining portion 66. As shown in FIGS. 5, 8, and 10, a slot 68 for receiving the tube strap 64 is formed across the arm 62. The slot 68 has chamfered or recessed entries 70 on either side so that the strap 64 can be threaded through the slot in either direction and the retaining portion 66 can seat in one of the chamfered entries 70, fixing that end of the strap to the arm 62. A free length of the tube strap 64 extends in a direction transverse to arm 62 and can be wrapped around the ET tube 44 as shown in FIG. 1. An adhesive pad 32 (not shown) or layer, such as a suitable pressure-sensitive adhesive, can be provided on an inner surface of the tube strap 62 to further restrain the ET tube 44 from rotational or longitudinal movement when secured against the bottom of the arm 62. A plurality of short but sharp, nub-like tangs or spikes 72 can also be provided on the bottom surface 74 of the arm 62. The spikes 72 can impinge on the exterior surface of the ET tube 44 to frictionally and/or physically engaging the ET tube and further restraining the tube from movement.

A clamping means is provided on a top side 75 of the arm 62, opposite the bottom side 74. The clamping means in the disclosed example is substantially similar to that disclosed in the aforementioned '504 patent. The clamping means is configured to securely engage and clamp a segment of the tube strap 64 along its free length, also shown in FIG. 1. The clamping means can maintain the tube strap 64 under tension to further help restrain the ET tube 44 from unintended movement. In the disclosed example, the clamping means is an elongate, cantilevered clamping lever 76 that is attached via a living hinge 78 to the tube holder 40, as shown in FIGS. 8 and 10. The clamping lever 76 can be pivoted and raised about the living hinge 78. The free length of the tube strap 62 can be folded over the top side 75 of the arm 6. The clamping lever 76 can be lowered and locked in place against the arm 62 to pinch and maintain the tube strap 62 in tension about the ET tube 44. A lengthwise or axial rib 80 is provided in this example and protrudes down from the clamping lever 76. The top side 75 of the arm 62 has a corresponding longitudinal or lengthwise channel or recess 82 sized to receive the rib 80 when the clamping lever is closed. The rib 80 and channel 82 are provided in order to crimp the tube strap 62 and help to ensure that the tube strap does not slip from between the arm 62 and the clamping lever 76.

In this example, a latching means is also provided to lock and hold the clamping lever 76 in the locked or clamped condition of FIG. 1. The latching means in one example can have a flexible, resilient catch 84 provided on the top side 75 and near the front end of the arm 62. When the clamping lever 76 is lowered onto the top side 75 of the arm 62, the catch 84 will flex forward to permit the front edge 86 of the clamping lever 76 to snap past the catch 84. The catch 84 then captures and retains the clamping lever 76 in the closed or clamped position of FIG. 1 to tightly engage the tube strap 62. To release the ET tube 44, a person can pull the catch 84 forward away from the clamping lever 76 until the front edge 86 can clear the catch. The clamping lever 76 can then be raised to release the tube strap 62. This type of latching means allows one to release the ET tube 44, readjust its position, and then re-secure the tube again without having to replace any components, tape, straps, and the like.

The arm 62 is connected to the shuttle 60 by a flexible leg 90. The flexible leg 90 in this example has one or more relieved sections 92 that allow the leg to bend and flex so that the arm 62 can move slightly relative to the shuttle. Such flexibility can impart a degree of give or yield between the shuttle and the arm so that the track can stay in position on the patient's face even while the patient's involuntary movements might cause movement of the ET tube 44 and the arm 62 during use.

As will be evident to those having ordinary skill in the art upon reading this disclosure, the disclosed device 30 is not to be limited to the particular tube holder construction disclosed herein. The arm, latching means, clamping means, flexible leg, and shuttle can vary in configuration and construction and yet function as intended.

The positioning mechanism 42 in the disclosed example is substantially similar to that disclosed in the aforementioned '504 patent. In general, the positioning mechanism has two components with one being carried on the track 32 and the other being carried on the shuttle 60 of the tube holder 40. The positioning mechanism 42 in this example allows selective lateral positioning of the tube holder 40 and the ET tube 44 along the track 32 without having to remove the device 30 from the patient or the ET tube from the device. In this example, one component of the positioning mechanism is a rail 94 disposed on the exposed side 36 of the track 32. The rail 94 is generally T-shaped in cross-section (i.e., when viewed from the side of the device 30) as shown in FIG. 2. The rail 94 is complementary shaped relative to a back side of the shuttle 60. In this example, the shuttle 60 has a C-shaped sliding retainer 96 that slides along, captures, and engages the rail. The T-shaped rail 94 and C-shaped retainer 96 can fit snuggly to one another but without impeding lateral sliding movement of the shuttle 60 along the track 32. One or more stops (not shown) may be provided at each of the opposite ends 38 of the track 32 to keep the shuttle 60 from sliding off either end of the rail 94 during use. These stops can allow the shuttle 60 to be snapped onto one end of the track 32 during assembly of the device 30.

The positioning mechanism also includes a locking means, in this example also partly on the shuttle 60 and partly on the track 32, to positively lock and hold the tube holder 40 and ET tube 44 in the selected position of adjustment on the track. In one example, as shown in FIGS. 1-4, the locking means has a pair of dogs or flex fingers 100 oriented generally perpendicular to the track 32. The flex fingers 100 are resiliently connected to the shuttle 60 and have prongs 102 that project rearward toward the track 32. The prongs 102 are positioned to engage any one of a plurality of ratchet teeth 104 provided on a front face of the rail 94. When the flex fingers 100 are in an unflexed condition, the prongs 102 seat in recesses 106 between the teeth 104. Grip ends 108 on the flex fingers 100 can be squeezed together, which spreads the flex fingers outward away from one another and disengages the prongs 102 from the ratchet teeth 104. When disengaged, the tube holder 40 and shuttle 60 can slide along the rail 94 to a desired position along the track 32. This can be done to allow medical personnel to access the patient's mouth without having to remove the device 30 or the ET tube 44. When pressure on the grip ends 108 the flex fingers 100 is released, the prongs 102 will return and engage the ratchet teeth 104 to retain the tube holder 40 in the selected position along the track 32.

As will be evident to those having ordinary skill in the art, the positioning mechanism components, including the locking means, the rail, and the shuttle retainer, can vary from the example shown and described herein. The details of the structure and function of these aspects of the device 30 can be varied and yet function as intended. The device 30 has a tube holder 40 that is side-to-side adjustable along the track, even when the device is attached to a patient. Thus, the ET tube 44 can be moved from one side to the other within the patient's mouth to allow medical personnel to clean the patient's teeth or attend to other medical care without having to completely remove the device 30 from the patient and without having to remove the ET tube 44 from the device. This makes caring for the patient simpler and easier.

According to the teachings of the present invention, the bite block 46 is integrated into the tube holder 40, as best illustrated in FIGS. 5-11. In one example, the bite block 46 can be integrally molded from a suitable plastic or similar material as a part of the tube holder 40. This can render the entire tube holder 40 a one-piece unit inclusive of the bite block 46, the arm 62, the shuttle 60, the flex fingers 100, and the clamping mechanism for the tube strap. In one alternate example, the bite block 46 could be a separate element that is configured to attach to the tube holder. This could be done by utilizing a complementary snap connection between bite block and holder or by utilizing an adhesive, fasteners, or the like. In such an example, the bite block could be utilized on a patient where the medical personnel determine such usage beneficial. The bite block could also be removed in such an example if the medical personnel determined that the bite block should not be used for some reason.

In the disclosed example, the bite block 46 is molded to a rear end 110 of the arm 62 opposite the catch 84. A molded joint 112 connects the bite block 46 to the arm 62 in this example. The molded joint 112 can vary in configuration and construction. The intent is for the connection to be robust and durable so that the bite block 46 remains attached to the tube holder 40, as long as intended. The joint 112 can also be positioned and configured to connect the bite block 46 to the tube holder 40 at a number of different locations on both the bite block and holder. In this example, the molded joint 112 is on the rear end 110 of the arm 62 and connects to a forward end 114 of the bite block 46. Also in this example, the bite block 46 is positioned spaced vertically below the track 32. In other configurations, as noted above, it is possible that the bite block be positioned and spaced above the track. The positioning of the bite block 46 in this example is such that the ET tube 44 can still be retained in place by the tube strap 64 and by the spikes 72, which are still exposed forward of the bite block on the bottom side 74 of the arm 62.

Features of the bite block 46 are depicted in FIGS. 5, 6, and 8. The bite block 46 has a generally tubular wall 120 having a substantially cylindrical shape with a length and a central axis. A central opening 122 extends along the length of the bite block 46 within the wall 120 and the wall has a pair of opposite open ends including the aforementioned forward end 114 and a rear or working end 126. The working end portion of the wall 120 of the bite block extends in the direction of the face contacting side 34 of the track 32, i.e., in a rearward direction and extends further rearward beyond the surface or face of the face contacting side.

In the disclosed example, the bite block 46 has an insertion slot 128 through the wall 120. The insertion slot 128 extends along the entire length of the bite block 46. The wall 120 can be formed of a suitable plastic material so that the bite block 46 is sufficiently flexible to allow an ET tube 44 to be inserted laterally into the central opening 122 through the insertion slot 128. The flexibility of the wall 120 can allow the width of the slot 128, and thus the diameter of the wall, to expand when inserting the tube and then to spring back to the normal at rest slot width and wall diameter once the ET tube 44 is fully inserted. The insertion slot 128 is defined between spaced apart free edges 130 (upper) and 132 (lower) of the wall 120. The free edges 130, 132 confront one another along the length of the bite block 46 and define the width of the slot 128. The wall 120 also has an optional blind slot 134 that is formed in the working end portion of the bite block 46. The blind slot 134 is open at the working end 124 of the wall 120 and terminates only part way along the wall at a closed or blind end 136. Thus, unlike the insertion slot 128, the blind slot 134 extends only part way along the length of the wall 120. The blind slot is also positioned generally opposite the insertion slot 128 in the wall. The blind slot 134 can add some resilience and flexibility to the wall 128. Thus, during use, the wall 120 can give slightly if a patient were to occasionally exert a great amount of force upon the bite block 46 during use. This would divert some of the load or absorb some of the energy from such a force through the wall 120 instead of directly to the patient's jaw and/or teeth.

The bite block 46 can also include one or more features to accommodate accessory lines passing through the bite block adjacent the ET tube 44. Such an accessory line feature can extend the length of the bite block and be formed between the wall and the ET tube within the central opening of the bite block. In this example, the bite block 46 has an accessory line channel 138 that extends the length of the bite block 46 within the central opening 122. The accessory line channel 138 can be formed, at least in part on an inner surface 140 of the wall 120. The accessory line channel 138 can also be formed in part by the outer surface of the ET tube 44, depending on the configuration and construction of the channel. In this example, the accessory line channel 138 is formed between the inner surface 140 of the wall 120 and the outer surface of the ET tube 44. Two spaced apart ribs 142 extend along the length of the bottom of the bite block 46 on the inner surface 140 of the wall 120. The channel 138 is defined between the ribs 142 and between the outer ET tube outer surface and the inner surface 140 of the wall 120. The ribs 142 create a space between the inner surface 140 and the ET tube 44. An accessory line, such as a pilot tube 144 (see FIG. 1) for inflating a retention cuff or balloon (not shown) on the end of the ET tube 44, can be run along the outside of the ET tube 44 and through the bite block 46 along the channel 138 without the line being pinched off, closed, or crushed.

More than one accessory line channel can be formed along the inside of the bite block 46. Three or more spaced apart ribs can be provided to define two or more separate channels, if desired. Also, two or more accessory lines can be run along the same channel, if desired. The accessory lines can provide any type of additional line function needed to treat a patient, including but certainly not limited to the pilot line 144. Also, the configuration, construction, and performance features of the bite block can vary from the bite block 46 described above. For example, the free edges 130, 132 of the insertion slot 128 on the bite block 46 can be varied in shape and contour so as to help the wall 120 retain a generally cylindrical shape, even when a patient's teeth exert a substantial crushing force on the bite block during use. The upper free edge 130 could have a first contoured shape and the lower free edge 132 could have a corresponding second contoured shape configured to close against and engage the first contoured shape if a sufficient clamping force is exerted on an outer surface of the bite block.

For example, FIGS. 12 and 13 show a tube holder 150 that is substantially identical to the earlier described tube holder 40. In this example, the tube holder 150 has a bite block 152 that also has a tubular shape with a substantially cylindrical wall 154. However, the bite block 152 has a simpler configuration with no accessory line channel and no blind slot. The bite block 152 does have an insertion slot 156 along a length of the slot and through the wall 154. Free edges 158, 160 of the insertion slot 156 are contoured in this example and are closer together resulting in a narrower insertion slot 156 than the slot 128 of the bite block 46. The upper free edge 158 has an angled or beveled edge forming a flat face 162 and the lower free edge 160 has a corresponding angled or beveled edge defining a complementary flat face 164. If a patient were to apply sufficient bite force on the bite block, the flat faces 162, 164 would engage and mate with one another under less of a load than the earlier disclosed bite block 46 because the free edges 158, 160 are closer together. Also, the flat faces 162, 164 being in contact will help prevent the free edges 158, 160 from bypassing one another and thus will help prevent the wall 154 from collapsing beyond its cylinder shape.

FIGS. 14 and 15 show another example of a tube holder 170 that is also substantially identical to the earlier described tube holder 40. In this example, the tube holder 170 has a bite block 172 that also has a tubular shape with a substantially cylindrical wall 174. The bite block 172 also has a simpler configuration with no accessory line channel and no blind slot in comparison to the bite block 46. The bite block 172 does have an insertion slot 176 along a length of the slot and through the wall 174. Free edges of the insertion slot 176 are also contoured in this example. Forward edge portions 178a, 180a are not contoured and are further apart from one another leaving a greater slot width at a forward end of the insertion slot 176. Rearward edge portions 178b, 180b of the free edges are closer together resulting in a narrower width slot over part of the insertion slot 176 than the slot 128 of the bite block 46.

The upper free edge portion 178b has an angled or beveled edge forming a flat face 182 and a convex V-shaped terminus 184 along the free edge portion. The lower free edge portion 180b has a corresponding angled or beveled edge defining a complementary flat face 186 and also has a stepped ridge 188 protruding into the central opening 122. The stepped ridge cooperates with the complementary flat face 186 to create a concave V-shaped trough or groove 190 along the free edge portion 180b. If a patient were to apply sufficient bite force on the bite block 170, the bottom terminus 184 would engage and seat in the groove 190 to help prevent the two free edges 178b, 180b from bypassing one another and thus to help prevent the wall 172 from collapsing beyond its cylinder shape. The contoured free edge portions in this example may be more robust in this regard that those of the bite block 150.

FIGS. 16 and 17 show another example of a tube holder 200 that is also substantially identical to the earlier described tube holder 40. In this example, the tube holder 200 has a bite block 202 that is substantially the same as the bite block 170, other than the contoured edge portions. In this example, the bite block 202 also has a tubular shape with a substantially cylindrical wall 204. The bite block 202 also has no accessory line channel and no blind slot in comparison to the bite block 46. The bite block 202 does have an insertion slot 206 along a length of the slot and through the wall 204. Free edges of the insertion slot 206 are also contoured in this example. Forward edge portions 208a, 210a are not contoured and are further apart from one another leaving a greater slot width at a forward end of the insertion slot 206. Rearward edge portions 208b, 210b of the free edges are closer together resulting in a narrower width slot over part of the insertion slot 206 than the slot 128 of the bite block 46.

The upper free edge portion 208b has a rounded convex contour forming a rounded face 212 along the free edge portion. The lower free edge portion 210b has a flange 214 that is wider than the wall 204 thickness. The upward facing side of the flange 214 has a concave rounded groove sized to complement the rounded face 212 of the upper edge portion 208b. If a patient were to apply sufficient bite force on the bite block 200, the rounded face 212 would engage and seat in the rounded groove 216 to help prevent the two free edges 208b, 210b from bypassing one another and thus to help prevent the wall 202 from collapsing beyond its cylinder shape. The contoured free edge portions in this example also may be more robust in this regard that those of the bite block 150. In these examples, the wider insertion slot portion can help a medical technician to start insertion of the ET tube 44 into the bite block before having to further spread apart the remainder of the insertion slot to complete insertion of the ET tube.

The bite block in other examples can have alternative concave and convex complementary shapes, or other complementary contoured shapes that can engage and mate with one another. The invention is not limited only to these examples described herein.

For example, FIGS. 18 and 19 show a tube holder 220 that is also substantially identical to the earlier described tube holder 40. In this example, the tube holder 220 has a bite block 222 that also has a tubular shape with a substantially cylindrical wall 224. The bite block 222 has a simpler configuration with no blind slot in comparison to the tube holder 46. The bite block 222 does have an insertion slot 226 along a length of the slot and through the wall 224. Free edges 228, 230 of the insertion slot 226 are contoured in this example and are again closer together resulting in a narrower insertion slot 226 than the slot 128 of the bite block 46. Both the upper free edge 228 and lower free edge 230 have mirror image flanges 232, 234 that extend radially inward into the central opening of the bite block 220.

The flanges 232, 234 make the free edges 228, 230 wider than the wall 224 thickness. The flanges 232, 234 are configured to close against and engage one another when a sufficient clamping force is exerted on the bite block, which is less than that of the bite block 46, again because the free edges 228, 230 are closer together. Also, the flanges 232, 234 being wider than the thickness of the wall 224 will help prevent the free edges 228, 230 from bypassing one another and thus will help prevent the wall 224 from collapsing beyond its cylinder shape. Each of the flanges 232, 234 in this example also creates an accessory line channel 236, 238 along an inner surface 240 of the wall 224 adjacent the flanges within the central opening of the bite block 220. It is possible that only one of the flanges is configured to form or define such an accessory line channel.

FIGS. 20 and 21 show another alternate example of a tube holder 250 that is substantially identical to the earlier described tube holder 40. In this example, the tube holder 250 has a bite block 252 that also has a tubular shape with a substantially cylindrical wall 154. The bite block 252 also has no accessory line channel and no blind slot. The bite block 252 does have an insertion slot 256 along a length of the slot and through the wall 254. Free edges 258, 260 of the insertion slot 256 are spaced apart and contoured in a manner that is essentially identical to the free edges 158, 160 on the bite block 152 of the tube holder 150 described previously. The bite block 250 also has a living hinge 266 or relief in the wall 254 generally opposite the insertion slot 256 and extending a length of the bite block. The living hinge can be formed in a thickened portion 268 of the wall as shown. Alternatively, the entire wall can be thicker, other than the living hinge 266. The living hinge can be provided to allow for easier flexing of the wall to insert the ET tube. The thicker wall material can provide a more robust bite block while the living hinge still permits easy ET tube insertion via the insertion slot.

FIGS. 22 and 23 show yet another example of a tube holder 270 with features similar to earlier described bite block features. In this example, the tube holder 270 has a bite block 272 with two sets of ribs 274 forming two accessory line channels, one above the ET tube and one below the ET tube. The ribs 274 are otherwise similar to the ribs 142 described previously. The bite block 272 also has a living hinge 276 similar to the living hinge 266 in the previous example. Also in this example, free edges 278 of the bite block 272 along an insertion slot 279 can be configured to contact one another upon compression of the bite block. In this example, the free edges 278 can also be configured so that one of the free edges slips past the other until it contacts one of the ribs 274, which can act as a stop to prevent further compression.

FIGS. 24 and 25 show still another alternate example of a tube holder 280 with features similar to earlier described bite block features. In this example, the tube holder 280 has a bite block 282 with an insertion slot 284 similar to the slot 128 of the bite block 46, but with a wider insertion slot width. This bite block 282 has no blind slot opposite the insertion slot 284 but does have two spaced apart ribs 286 forming a single accessory line channel within the bite block.

FIGS. 26 and 27 show still another alternate example of a tube holder 290 with most features similar to earlier described bite block features. In this example, the tube holder 290 has a bite block 292 with an insertion slot 294 similar to the slot 128 of the bite block 46 with a wide slot width. This bite block 282 also has a blind slot 296 similar to the blind slot 134 on the bite block 46 and positioned opposite the insertion slot 294. The bite block 292 has a groove 298 formed along a length of the bite block and recessed into an inner surface 300 of the bite block wall 302. The groove 298 creates an accessory line channel that is recessed into the wall 302, instead of using protrusions to create space between the inner wall and an ET tube in the bite block, as in prior examples. The groove can be V-shaped in cross-section or can have a rounded, semispherical, or other shape as well. The groove 298 creates a thinner wall segment of the wall 302 as shown in FIG. 27. This thinner wall segment can provide dual functions of creating an accessory line channel as well as creating flex relief in the wall to aid in inserting the ET tube into the bite block.

FIGS. 28-30 show yet another example of a tube holder 320 that is nearly identical to the tube holder 270 described above. In this example, the tube holder 310 has a bite block 322 with two sets of ribs 324 and 326 forming two accessory line channels, one above the ET tube and one below the ET tube. In this example, the upper ribs 326 are longer than the lower ribs 324. The longer upper ribs provide a deeper accessory line channel between them. In one example, as shown in FIG. 30, the accessory line can be a subglottic suction line 328 that also extends along and communicates with the ET tube. Such a tube may have a larger diameter than a conventional pilot line and require a deeper channel within the bite block. When the bite block 322 is compressed during use, the rib length can determine how much crush is imparted on the accessory line, such as the subglottic suction line and how much crush is imparted on the ET tube.

FIGS. 31-33 show still another example of a tube holder 330 that is substantially the same as many of the prior described example. In this example, the tube holder 330 has a bite block 332 that is substantially the same as the bite block 46. However, the bite block has a second scallop or blind slot 334 on the front end of the bite block and opposite the insertion slot 336. The tube holder 330 also has a revised joint 336 that extends the bite block 332 a bit more rearward from the arm 340 than prior described tube holders. This extended joint 336 in combination with the blind slot 338 creates a gap or clearance 342 under the arm 340 and forward of the bite block 332 for routing an accessory line. As shown in FIG. 33, a second accessory line 344, such as a subglottic suction line, can exit the bite block adjacent the ET tube and pass under the arm 340 and joint 336.

FIGS. 34-36 show still another example of a tube holder 350 that is substantially the same as many of the prior described example. In this example, the tube holder 350 has a bite block 352 that has a raised recess or channel 354 formed in the upper surface of and extending the length of the bite block 352. The channel 354 create a larger sized passage within the bite block 352 for an accessory line without significantly affecting the size of ET tube that the bite block 352 can handle. As shown in FIG. 36, a second accessory line 356, such as a subglottic suction line, can enter the bite block adjacent the ET tube and pass within the channel 354 along with the ET tube.

FIG. 37 shows another example of a tube holder 360 that is essentially the same as the tube holder 40 described earlier and has a substantially similar bite block 362. However, in this example, the bite block 362 has a softer pad 364 provided on the top and bottom of the bite block 362. The pads can be adhered to the exterior of the bite block or can be in-molded or dual molded with the bite block but from a softer material. The soft pads 364 can be added for patient comfort and perhaps safety. The forces applied to the bite block 362 during use would be at least partly dispersed across the surface of the block via the softer pads 354. This can minimize stress concentration on the barrel of the bite block and help reduce the likelihood of damage to the patient's dentin while also increasing comfort for those that are awake and actively biting. In another example, the pads 364 could be combined with the taller bite block 352 on top of the channel 354 from the immediately prior example to increase comfort for the patient. The soft material pads 364 in such an example could be over-molded to cover the raised area of the channel 354 for the subglottic suction line 356 or other accessory line to create a soft landing for the patient's upper incisors.

In still another example as shown in FIGS. 38 and 39, a tube holder 366 that is substantially the same as the earlier described bite block 270 of FIGS. 23 and 24, with shorter upper ribs 367 can be used, but have an increased diameter bite block 368. The tube holder 366 can be used in conjunction with a larger sized accessory line 370, such as a subglottic suction tube. The ribs 367 and line 370 can be configured to allow for a predetermined amount of compression or crush on the line. The bite block 368 can also be configured to allow for a predetermined amount of compression or crush on the ET tube 372. A shown in FIG. 39, and as discussed above, the bite block 368 can be configured to allow for some compression before the free ends 374 along the insertion slot 376 come in contact with one another to resist further compression. The living hinge 378, similar to the earlier described hinges, can help with flexibility in the bite block 368 to insert the ET tube 372 and can also help with allowing some limited compression against reduced resistance from the wall.

Any number of the bite block and tube holder features described above can be used in combination, even though such combination is not specifically mentioned herein. Also, the disclosed features can be altered or modified from the examples shown and described above and yet function as intended. For example, the blind slot can be added in combination with other of the bite block embodiments and features disclosed and described herein. The blind slot can also be eliminated in any of the disclosed examples. The blind slot can be positioned on the forward end of the bite block and not on the rearward end as shown in FIGS. 5, 8, and 10. Likewise, the depth or length of the blind slot and/or the width or height of the blind slot can be altered to achieve a desired amount of flex of the bite block and at the desired position on the bite block. In one example, FIG. 40 shows a tube holder 380 with a bite block 382 having a blind slot 384 formed in a forward end 386 of a wall 388 of the bite block. In another example, FIG. 41 shows a tube holder 400 with a bite block 402 having a blind slot 404 that is similar to that in FIGS. 5, 8, and 10, but with a shorter length L to the blind end 404 and a smaller width W or height of the slot.

In another example, FIG. 42 shows an end view of a bite block 420 that is part of a tube holder (not shown) as previously described. The bite block 420 has a wall 422 with an insertion slot 424 and bottom rails 426 that create an accessory line channel 428 between the rails, also as previously described herein. The bite block 420 in this example is not completely round or cylindrical, but instead has a domed top segment 430 that creates another accessory line channel 432 at the top of the central opening within the bite block 420. The dome shaped of the wall top segment 430 creates a space between the ET tube (not shown) and the interior wall surface 434 that forms the channel 432. In this example, the wall has a consistent thickness and is instead dome shaped at the top to create the upper channel 432. In contrast, the earlier described bite block 292 in FIGS. 26 and 27 employs a recess in the interior surface of the bite block 292 that creates a thinner wall section. The result is a substantially similarly or identically shaped accessory line channel (though at the bottom of the bite block 292).

In another example, FIG. 43 shows an end view of a bite block 440 that is part of a tube holder (not shown) as previously described. The bite block 440 has a wall 442 with an insertion slot 444 and bottom rails 446 that create an accessory line channel 448 between the rails, also as previously described herein. The bite block 440 in this example also has a single upper rail 450 protruding down from an interior surface 452 near the top of the bite block. The single upper rail can create a space between the interior surface 452 and the ET tube (not shown) at least adjacent each side of the rail. The spaces can thus create additional accessory line channels 454 near the top of the bite block, or elsewhere in the bite block, within the central opening of the bite block. The additional accessory channels 454 are similar to the accessory channels 236, 238 created by the flanges 232, 234 on the bite block 222 in FIGS. 18 and 19 described above.

In another example, FIG. 44 shows an end view of a bite block 460 that is part of a tube holder (not shown) as previously described. The bite block 460 has a wall 462 with an insertion slot 464 and bottom rails 466 that create an accessory line channel 468 between the rails, also as previously described herein. A wall segment 470 between the bottom rails 466 has a thinner wall thickness in comparison to the remaining portions of the wall 462. The thinner wall thickness creates a two-fold function. First, the wall 462 is thinner on the interior side or surface 472 creating a recess in the wall, substantially similar to the groove or recess 298 of the bite block 292 describe earlier and shown in FIGS. 26 and 27. The greater depth creates a deeper accessory line channel 468 between the rails 466 without increasing the diameter of the bite block 460 or the height of the rails. Second, the thinner wall segment 470 creates a more flexible zone in the wall, as does the recess 298 in the bite block 292 in FIGS. 26 and 27, as well as the living hinge 266 shown in FIGS. 20 and 21 and the living hinge 276 shown in FIGS. 22 and 23. The flexible zone can make it easier to insert the ET tube into the bite block 460. The bite block 460 essentially combines the concepts of the bottom rails 286 in FIGS. 24 and 25 with the groove or recess 298 in FIGS. 26 and 27.

In another example, FIG. 45 shows an end view of a bite block 480 that is part of a tube holder (not shown) as previously described. The bite block 480 has a wall 482 with an insertion slot 484 and bottom rails 486 that create an accessory line channel 488 between the rails. The wall 482 has a wall segment 490 between the bottom rails 486. The wall segment 490 has a thinner wall thickness, identical to the bite block 460 of FIG. 44. The bite block 480 also has a wall segment 492 opposite the insertion slot 484 that is thicker than the remaining portions of the wall 482, similar to the bite block 252 in FIGS. 20 and 21. The thicker segment 492 can aid in resisting vertical crush of the bite block 480 while the thinner segment creates the accessory line channel 488 and a flex zone in the wall 482 to help with ET tube insertion into the bite block 480. The bite block 480 essentially combines the concepts of the thickened wall section 268 of the bite block in FIGS. 20 and 21 with the groove or recess and rail combination of the bite block 460 in FIG. 44. Also, the thinner wall segment 90 acts as the living hinge 266 of FIGS. 20 and 21, just in a different location on the bite block.

In another example, FIG. 46 shows an end view of a bite block 500 that is part of a tube holder (not shown) as previously described. The bite block 500 has a wall 502 with an insertion slot 504 and bottom rails 506 that create an accessory line channel 508 between the rails. The wall 502 has a wall segment 510 between the bottom rails 506. The wall segment 510 has a thinner wall thickness, which is again identical to the bite block 460 of FIG. 44. The bite block 500 also has an accessory line channel 512 at the top of the bite block 500, identical to the earlier described bite block 352 and channel 354 in FIGS. 34-37. The bite block 500 essentially combines the concepts of the large diameter accessory line channel 354 of the bite block 352 in FIGS. 34-36 with the groove or recess and rail combination of the bite block 460 in FIG. 44.

In another example, FIGS. 47 and 48 show a tube holder 520 with an integrated bite block 522 for an ET tube holding device such as the device 30. The tube holder 520 is substantially the same as the earlier examples, such as the tube holder 40, other than certain modifications to the bite block. In this example, the bite block 522 has a blind slot 524 formed in a forward end 526 of a wall 528 of the bite block. The blind slot 524 is similar to the tube holder 380 and bite block 382 of FIG. 40. However, in this example, the blind slot 524 is shorter in length or depth but taller in width or height in comparison to the blind slot 384 of FIG. 40. Also, the bite block 522 in this example includes an insertion slot 530 and bottom rails 532 that create an accessory line channel 534 between the rails. The wall 528 has a wall segment 536 between the bottom rails 532. The wall segment 526 has a thinner wall thickness, which is again identical to the bite block 460 of FIG. 44. The bite block 520 essentially combines the concepts of the forward end blind slot 384 of FIG. 40 with the groove or recess and rail combination of the bite block 460 in FIG. 44.

The foregoing examples are provided merely to further show that various combinations of and modifications to the bite blocks and features disclosed herein are within the spirit and scope of the present invention. Further modifications and feature combinations can also be made, though not specifically shown or described herein. For example, the size dimensions, shapes, wall thicknesses, contours, and the like of the features of the disclosed bite blocks can also be varied, depending on a particular application. Also, the bite block diameter can be changed, the insertion slot opening size or width can be changed, the wall thickness can be varied or changed, the rail heights can be changed, the rail positions can be altered, and the like.

Also, one or more additional or different features can also be added to any of the bite block examples disclosed herein to aid in inserting an ET tube into the insertion slot and to help prevent discomfort and irritation to the patient during use. As shown in FIG. 10 for example, the forward ends (or the rear ends, if desired) of the free edges 130, 132 on the bite block 46 can include angled or tapered entry segments 310, 312 at the leading edge of the insertion slot 128. 2The tapered entry segments 310, 312 can make the insertion slot 128 wider, but only at the forward end of the bite block 46. This can make it easier for medical personnel to spread apart the free edges 130, 132 in order to aid in starting to insert an ET tube 44 into the bite block 46. Such a feature can be included on any of the disclosed bite blocks and is shown on many of them herein.

In another example, the end face of the bite block need not lie in a plane. Instead, the end face can be curved to form rounded curved contours, especially on the working end of the bite block that will lie within a patient's mouth. For example, FIGS. 5 and 10 show that the bite block 46 has curved contoured regions 314 in the axial direction at the working end 124 of the bite block 46. Likewise, the exposed end edges of the bite block can also be smooth and curved or rounded. For example, an end edge 316 of the bite block 200 shown in FIGS. 16 and 17 are smooth and rounded instead of having sharp corners, particularly at the working end of the bite block. Such features can make the device 30 more comfortable for a patient during use.

The disclosed ET tube holding device 30 can be applied or installed on the patient with the ET tube 44 already positioned in the patient's mouth and trachea. If a temporary bite block device is already prepositioned about the tube, that bite block can be removed and the bite block as described herein can be attached to the ET tube at the same time that the device 30 is installed. The disclosed bite blocks can be constructed from materials and material thicknesses and with features that render the bite block sufficiently rigid to inhibit the inserted ET tube from being crushed or closed off by a patient's bite during use and yet sufficiently flexible to allow relatively easy insertion and removal of the ET tube as needed.

Also, each of the above-described examples includes a bite block that is integrally molded or provided as part of the ET tube holding device. As mentioned above, the bite block can instead be configured to be selectively attachable to and even optionally detachable from a part of the device, such as the tube holder. For example, FIGS. 49 and 50 show one possible embodiment of a tube holder 550 with a separately attachable and optionally detachable bite block 552, each constructed in accordance with the teachings of the present invention. As with the prior examples, the tube holder 550 and bite block 552 are configured for use on an ET tube holding device such as that described herein. The tube holder and/or the bite block of this example can, however, be used with other holding device configurations and constructions as well.

In this example, the tube holder 550 has an arm 554 with a rear facing surface 556. A connector 558 is provided on the surface 556. The connector 558 can vary in construction and configuration. In this example the connector 558 is an elongate T-shaped keyed rib that protrudes laterally across the surface 556 of the arm 554. The bite block 552 has a mating connector 560 that is shaped to receive and engage the connector 556. In this example, the mating connector 560 is a recessed slot or channel that extends laterally across the forward end of the bite block. The channel or slot is open at each end or at least one end and also has a T-shape that matches the shape of the connector 558.

FIG. 49 shows the tube holder 550 and the bite block 552 separated from one another. FIG. 50 shows the two components connected to one another. In this example, the two components are assembled by sliding the bite block 552 laterally onto the tube holder 550 so that the T-shaped rib is received within the T-shaped channel. The bite block 552 can be selectively attached to or detached from the tube holder in this example, depending on whether the medical technician intends to utilize the bite block for a particular patient. To detach the bite block 552 from the tube holder 550, the technician can slide the bite block laterally until the keyed rib is free of the channel or slot.

FIGS. 51-53 show another example of a tube holder 570 with a detachable bite block 572, each constructed in accordance with the teachings of the present invention. As with the prior examples, the tube holder 570 and bite block 572 are configured for use on an ET tube holding device such as that described herein. In this example, the tube holder 570 has an arm 574 with a rear facing surface 576. A connector 578 is again provided on the surface 576. In this example the connector 578 is a pair of elongate flexible rods 580 that protrudes rearward from the surface 576 of the arm 574. The bite block 572 has a mating connector 582 that is shaped to receive and engage the connector 576. In this example, the mating connector 582 is a pair of through bores 584 that extend lengthwise along a top of the bite block through the bite block tubular wall 586 as shown in FIGS. 51 and 52. In this example, a top segment 588 of the tubular wall 586 is thicker than a thickness of the wall material adjacent the top segment to accommodate the through bores 584. Each rod 580 has a nubbin or button 590 at the free end that is slightly larger in diameter that the shaft of the rod 580.

FIG. 51 shows the tube holder 570 and the bite block 572 separated from one another. FIG. 53 shows the two components connected to one another. In this example, the two components are assembled by sliding the bite block 572 onto the flexible rods 580. The through bores 584 are sized to receive the pair of rods 580. The nubbins or buttons 590 can be forced along the through bores 584 and can pop out the other end of the bores. The nubbins or buttons 590 help to retain the bite block 572 attached to the rods 580 and thus the tube holder 570 in this example. The nubbins or buttons 590 can be one-way devices design to prevent reverse sliding back through the through bores 584. Alternatively, the nubbins or buttons 590 can permit forced reverse sliding back through the bores. In such a case, to detach the bite block 572 from the tube holder 570, the bite block can be pulled rearward from the rods 580. The nubbins or buttons 590 can be forced in a reverse direction through and out of the through bores 584 to release the bite block from the rods.

In this example, the rods 580 can be integrally molded as part of the tube holder and protruding from the surface 576. Alternatively, the flexible rods 580 can be separately fabricated and then attached to the tube holder 570. The rods 580 and the respective nubbins or buttons 590 can each also be formed as one integral piece, such as from molded plastic or other suitable materials.

FIGS. 54-57 depict another example of a tube holder 600 with a detachable bite block 602, each constructed in accordance with the teachings of the present invention. This example combines the concept of the two prior examples into one product. As with the prior examples, the tube holder 600 and bite block 602 are configured for use on an ET tube holding device such as that described herein.

The tube holder 600 in this example has an arm 604 with a rear facing surface 606. A connector 608 is provided on the surface 556. In this example the connector 608 is an elongate T-shaped keyed rib that protrudes laterally across the surface 606 of the arm 604, in the same manner as the connector 554 described above and shown in FIGS. 49 and 50. An intermediate joint 610 has a mating connector 612 that is shaped to receive and engage the connector 606. In this example, the mating connector 612 is a recessed slot or channel that extends laterally across the forward end of the intermediate joint 610. The channel or slot is open at each end and also has a T-shape that matches the shape of the connector 608.

The intermediate joint 610 in this example is a curved strip of material with one or more relieved regions 614 across a width of the strip. The relieved regions 614 can impart resilient flexibility to the strip of material, which can be plastic or other suitably resilient, flexible materials. The channel or slot of the mating connector 612 is formed in a thicker portion 616 carried on one end of the intermediate joint in this example.

In this example, the intermediate joint 610 carries a second connector 618 at the other end of the strip of material. In this example the second connector 618 is a pair of elongate flexible rods 620 that protrudes rearward from the end of the strip of material. The rods 620 are essentially the same as the rods 580 in FIGS. 51 and 53. The bite block 602 has a mating second connector 622 that is shaped to receive and engage the second connector 618. In this example, the mating second connector 622 is a pair of through bores 624 that extend lengthwise along a top of the bite block through the bite block tubular wall 626 as shown in FIG. 55. The through bores 624 and bite block 602 can be the same as the bite block 572 shown in FIGS. 51-53 or can be of a different construction. In this example, the bite block 602 is different in that the tubular side wall forms a somewhat square cross-section shape on the exterior of the block while a central opening 628 through the bite block can be round or even non-round in cross-section.

FIGS. 54-56 show the tube holder 600, the intermediate joint 610, and the bite block 602 separate from one another. FIG. 57 shows the three components connected to one another. In this example, the three components are assembled by sliding the intermediate joint 610 laterally onto the tube holder 600 so that the T-shaped rib is received within the T-shaped channel. The intermediate joint 610 can be selectively attached to or detached from the tube holder 600, depending on whether the medical technician intends to utilize a bite block for a particular patient. To detach the intermediate joint 610 from the tube holder 600, the technician can slide the joint laterally until the keyed rib is free of the channel or slot. The bite block 602 is attached to the intermediate joint 610 by sliding the bite block onto the flexible rods 620. Nubbins or buttons 630 in this example can be forced along the through bores 624 and can pop out the other end of the bores. The nubbins or buttons 630 help to retain the bite block 602 attached to the rods 620 and thus the tube holder 600 in this example.

The rods and nubbins or buttons in each of the examples of FIGS. 51-53 and FIGS. 54-57 can be configured as one-way devices. In other words, a selected bite block can be attached to the rods, once attached, cannot be detached without destroying the ET tube holding device or at least the tube holder of these examples. Alternatively, the rods and buttons or nubbins (or some other retaining element) can be configured to permit forcibly detaching one bite block and replacing another bite block onto the rods.

Use of a bite block that can be selectively attached to an ET tube holding device allows the ability to providing a range of user selectable bite block sizes, materials, configurations, and the like. A specific bite block could be selected from a set of different bite blocks to accommodate specific ET or other tube criteria such as tube size, shape, tube material, tube hardness, and/or tube purpose or function. Also, the type of ET tube holding device can vary from the examples shown and described herein. The device may not employ a separate or adjustable tube holder. Instead, the bite block or the intermediate joint part can be attachable to and optionally detachable from another part of the device. FIGS. 54-57 are provided also to show that the bite block form and construction can vary as well to meet particular patient or critical care needs and applications.

Although certain ET tube holding devices, features, components, and methods have been described herein in accordance with the teachings of the present disclosure, the scope of coverage of this patent is not limited thereto. On the contrary, this patent covers all embodiments of the teachings of the disclosure that fairly fall within the scope of permissible equivalents.

What is claimed is:

1. A device for holding an endotracheal tube to a patient, the device comprising:
a track configured to fit adjacent a lip on a patient's face, the track having a face contacting side, an exposed side opposite the face contacting side, and a pair of opposite ends;
a tube holder coupled to and slidable along the track between the opposite ends;
a positioning mechanism that is releasably lockable to allow selective lateral repositioning of the tube holder and an endotracheal tube held thereby along the track and to retain the tube holder at a selected position along the track;
a bite block carried by the tube holder and slidable therewith along the track, the bite block having a tubular wall, a central opening along a length of the bite block, an interior wall surface, and a pair of opposite open ends, the bite block positioned spaced vertically from the track and one of the opposite ends being a working end portion that extends in the direction of and further beyond the face contacting side of the track; and
an accessory line channel positioned between the interior wall surface and an endotracheal tube that extends through the central opening.

2. A device according to claim 1, wherein the bite block has an insertion slot through the wall and along the entire length of the bite block, the wall being sufficiently flexible to allow an endotracheal tube to be inserted laterally into the central opening through the insertion slot.

3. A device according to claim 2, wherein the insertion slot is defined between spaced apart free edges of the wall that confront one another along the length of the bite block.

4. A device according to claim 3, wherein each of the free edges has a flange extending inward into the central opening of the bite block, each flange configured to close against and engage the other flange if a sufficient clamping force is exerted on an outer surface of the bite block.

5. A device according to claim 4, further comprising two of the accessory line channels, one being formed along the interior wall surface adjacent each of the flanges within the central opening of the bite block.

6. A device according to claim 3, wherein at least one of the free edges has a flange extending inward into the central opening of the bite block whereby the accessory line channel is formed along the interior wall surface adjacent the flange.

7. A device according to claim 2, further comprising a living hinge or relief in the tubular wall generally opposite the insertion slot and extending a length of the bite block.

8. A device according to claim 2, further comprising a blind slot formed in the tubular wall of the bite block and extending only part way along the length of the wall and positioned generally opposite the insertion slot in the wall.

9. A device according to claim 8, wherein the blind slot is formed in the working end portion of the bite block.

10. A device according to claim 8, wherein the blind slot is formed in the other of the opposite ends of the bite block.

11. A device according to claim 1, wherein the accessory line channel is formed between two spaced apart ribs extending the length of the bite block on the interior wall surface.

12. A device according to claim 11, wherein a wall segment of the tubular wall between the two spaced apart ribs is thinner than a thickness of the tubular wall.

13. A device according to claim 12, wherein the wall segment and the two spaced apart ribs are on the bottom side of the tubular wall and the wall segment is recessed into the interior wall surface of the tubular wall and being thinner than a thickness of the tubular wall adjacent the wall segment.

14. A device according to claim 1, wherein the accessory line channel is formed by a wall segment that is recessed into the interior wall surface, the wall segment being thinner than a thickness of the tubular wall adjacent the wall segment.

15. A device according to claim 14, wherein the wall segment is a V-shaped groove extending along and recessed into the inner surface of the wall.

16. A device according to claim 1, further comprising:
a cheek plate connected to each of the opposite ends of the track;
a cheek pad coupled to each of the cheek plates;
an adhesive layer on a face contacting side of each pad; and
an adjustable head strap coupled to the device for securing the device to a patient's head and retaining the track, cheek plates, and cheek pads on the patient's face.

17. A device according to claim 1, wherein the bite block is integrally molded as a part of the tube holder.

18. A device according to claim 1, wherein the bite block is selectively attachable to and detachable from the tube holder.

19. A bite block for an endotracheal tube, the bite block comprising:
a tubular wall with a pair of opposed ends;
a central opening along a length of the bite block between the pair of opposed ends;
an interior wall surface; and
an accessory line channel positioned between the interior wall surface and an endotracheal tube extending through the central opening and is formed by a wall segment that is recessed into the interior wall surface, the wall segment being thinner than a thickness of the tubular wall adjacent the wall segment.
